(19) United States
(12) Plant Patent Application Publication
(10) Pub. No.: US 2003/0172424 P1
(43) Pub. Date: Sep. 11, 2003

(54) Verbena plant named 'Lan Pureye'

(76) Inventor: Mitchell Eugene Hanes, Morgan Hill, CA (US)

Correspondence Address: JONDLE & ASSOCIATES P.C., 9085 EAST MINERAL CIRCLE, SUITE 200, CENTENNIAL, CO, 80112, US

(21) Appl. No.: 10/092,621

(22) Filed: Mar. 8, 2002

(51) Int. Cl. A01H 5/00

(52) U.S. Cl. PLT/308

(57) ABSTRACT

A verbena cultivar particularly distinguished by purple colored flowers, vigorous growth and low trailing habit.

### GENUS AND SPECIES

Verbena hybrida

### VARIETY DENOMINATION

‘Lan Pureye’

### BACKGROUND OF THE NEW PLANT

The present invention comprises a new and distinct cultivar of verbena, botanically known as Verbena hybrida, and hereinafter referred to by the cultivar name ‘Lan Pureye’. The new cultivar is asexually reproduced from vegetative cuttings and tissue culture resulting from the cross of the seed/pod parent 98-590-5, a blue proprietary line that is unnamed and unpatented×Temari Purple (PPAF), a purple commercial line.

‘Lan Pureye’ is a product of a planned breeding program intended to create new verbena cultivars with purple colored flowers, dark green foliage, vigorous growth and low trailing habit.

The new cultivar was created in 1999 in Gilroy, Calif. and has been asexually reproduced repeatedly by vegetative cuttings and tissue culture in Gilroy, Calif. over a one and a half year period. The plant has also been trialed at Gilroy, Calif., Litchfield, Mich. and Andijk, The Netherlands. The present invention has been found to retain its distinctive characteristics through successive propagations; and this novelty is firmly fixed.

### DESCRIPTION OF PHOTOGRAPH

This new verbena plant is illustrated by the accompanying photograph which shows blooms, buds, and foliage of the plant in full color, the colors shown being as true as can be reasonably obtained by conventional photographic procedures.

FIG. 1 shows overall plant habit;

FIG. 2 shows the mature inflorescence.

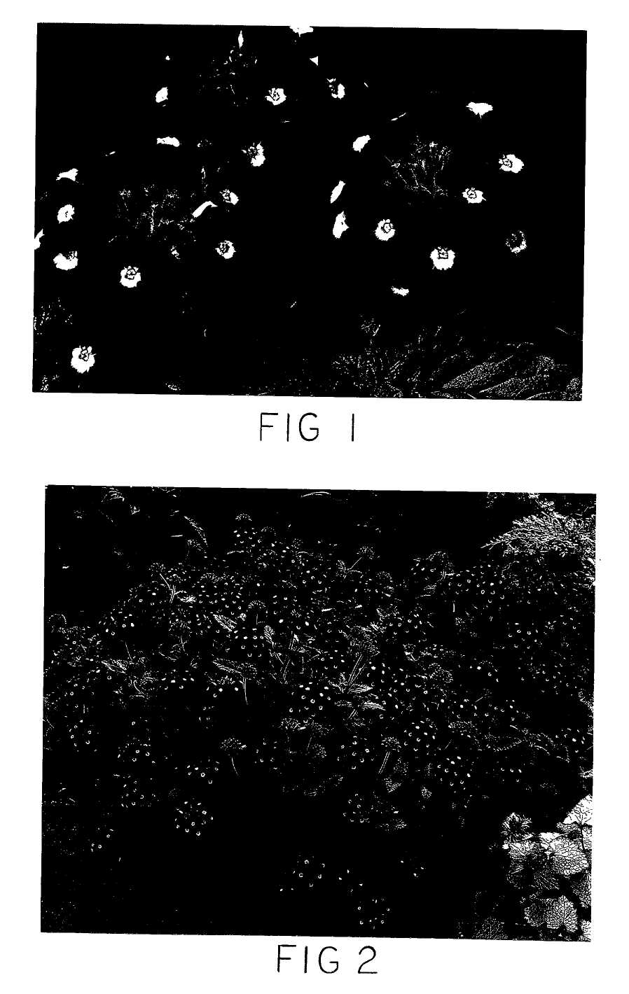

### DESCRIPTION OF THE NEW CULTIVAR

The following detailed descriptions set forth the distinctive characteristics of ‘Lan Pureye’. The data which defines these characteristics were collected from asexual reproductions carried out in Gilroy, Calif. The plant history was taken on 22 week old plants grown in one gallon pots, in a double poly-greenhouse under natural light and color readings were taken in the greenhouse. Color references are primarily to the RHS Colour Chart of The Royal Horticultural Society of London (RHS). Texture description details were observed under a dissecting microscope according to The New Royal Horticultural Society Dictionary of Gardening (1992).

### THE PLANT

Classification:

Botanical.—Verbena hybrida.

Commercial.—Verbena.

Form: Low trailing annual, decumbent

Growth and branching habit: Vigorous growing, moderate basal branching; low trailing habit

Height: From soil level to top of blooms: Approximately 23-25 cm

Width: Approximately 70-76 cm

Time to produce a finished flowering plant: 10 weeks.

Outdoor plant performance: Typical bedding plant culture, full sun in the garden, hanging baskets or container plants

Time to initiate roots: Approximately 4 days in the greenhouse

Time to develop roots: Approximately 7 days in the greenhouse

Root description: Fibrous, fleshy, white

### THE LEAVES

Length: 4.2-4.4 cm

Width: 2.1-2.2 cm

Leaf blade shape: Ovate/lanceolate

Leaf margin: Irregular, crenately incised

Apex aspect: Acute

Base aspect: Cuneate

Foliage color: Upper surface — Green RHS 147A; Lower surface is green RHS 147B.

Texture: Pilose; heavy on midvein with some glandular hairs

Venation: Pinnate

Venation color: Green RHS 145B

Petiole length: 0.3-0.4 cm

Petiole diameter: 0.15 cm

Petiole color: Green RHS 145B

### THE STEM

Length: 45-50 cm

Diameter: 0.2-0.3 cm

Internode length: 2.5-4.0 cm

Color: Yellow-green RHS 144A

Texture: Pilose (short and long), glandular hairs, some with violet color

Stem anthocyanin: None

### THE BUD

Shape: Linear

Diameter: 0.3 cm

Length: 1.4-1.5 cm

Color at tight bud: Purple RHS 83B

### THE FLOWER

Blooming habit: Continuous throughout the growing season

Inflorescence type: Spike

Spikes per plant: 100-110 in the greenhouse

Spike diameter: Approximately 4.5-5.5 cm

Spike depth: Approximately 3.0-4.5 cm

Peduncle length: 6.0-9.5 cm

Peduncle diameter: 1.5 cm

Peduncle color: Yellow-green RHS 144A

Peduncle texture: Many glandular hairs with violet color, short and long pilose.

Flower color: Upper petal surface is purple RHS 82A but brighter purple with a green RHS 145B “eye”; Lower petal surface is purple RHS 84A.

Floret form: Salverform; sessile on spikes

Floret (limb) diameter: Approximately 1.9-2.0 cm

Corolla tube length: Approximately 2.3-2.6 cm

Number florets per spice: 25-33 (closed to fully open florets)

Number of petals: Gamopetalous, five lobed

Petal size: Length of one lobe — 0.9-1.0 cm; width of one lobe — 0.8-0.9 cm

Petal lobe shape: Obcordate

Petal apex shape: Emarginate

Petal base shape: Fused

Petal margin: Entire

Petal texture: Papillose

Sepals: Five sepals whose margins are fused to each other along their length with a transparent membrane of less than 1 mm in width with one smaller sepal (5 mm) attached to the base of the calyx.

Calyx length: Approximately 1.0 cm

Calyx width: Approximately 2.0 cm

Calyx shape: Linear

Calyx apex: Acute

Calyx color: Yellow-green RHS 143B

Calyx texture: Short pilose, many glandular hairs with violet color

Lastingness of individual blooms: Moderate

Fragrance: None

### THE REPRODUCTIVE ORGANS

Stamens: Anthers and filaments fused to upper half of corolla tube; four anthers with two pollen sacs per anther.

Pollen amount and color: Moderate, yellow RHS 4A

Pistil: One style approximately 1.8-1.9 cm; slightly recurved at the stigma.

Stigma color: Green RHS 145A

Fruit seed set: Does not freely set seed but can produce 4 narrow nutlets, approximately 4 mm long; colored grey-brown RHS 199D and enclosed in the dried calyx.

### DISEASE AND INSECT RESISTANCE

Tolerant to mildew

### COMPARISON WITH KNOWN CULTIVARS

When the instant plant is compared to ‘Temari Burgundy’ (PPAF) the ‘Lan Pureye’ has leaves that are much larger than ‘Temari Burgundy’ more flowers per spike and has a flatter, lighter purple colored flower with a lighter colored “eye” while ‘Aztec Dark Purple’ is darker and has slightly ruffled petal margins. ‘Lan Pureye’ has a shorter pilose (pubescence) than ‘Aztec Dark Purple’.

I claim:

1. A new and distinct cultivar of verbena plant as shown and described herein.
